(19) United States
(12) Patent Application Publication
(10) Pub. No.: US 2023/0240621 A1
(43) Pub. Date: Aug. 3, 2023

(54) EVENT DETECTION IN SUBJECT SOUNDS

(71) Applicant: ResApp Health Limited, Brisbane, Queensland (AU)

(72) Inventors: Javan Tanner Wood, Brisbane, Queensland (AU); Vesa Tuomas Kristian Peltonen, Brisbane, Queensland (AU); John Campbell May, Brisbane, Queensland (AU); Nicholas Kim Partridge, Brisbane, Queensland (AU)

(73) Assignee: ResApp Health Limited, Brisbane, Queensland (AU)

(21) Appl. No.: 18/001,355

(22) PCT Filed: Jun. 18, 2021
(86) PCT No.: PCT/AU2021/050636

§ 371 Date: Dec. 9, 2022

(30) Foreign Application Priority Data
Jun. 18, 2020 (AU) 2020902025

(51) Int. Cl. A61B 5/00; A61B 7/00

(52) U.S. Cl. CPC A61B 5/7282; A61B 7/003; A61B 5/725; A61B 5/742; A61B 5/4818; A61B 2562/0204

(57) ABSTRACT

A method for identifying segments of a digital audio recording of sounds from a subject, where the segments contain particular sound events of interest, the method comprising: filtering the digital audio recording based on a characteristic frequency range of the sound events to produce a filtered digital audio signal; processing the filtered digital audio signal to produce a corresponding signal envelope; fitting a statistical distribution to the signal envelope; determining a threshold level for the signal envelope based on the statistical distribution and a predetermined probability level; and identifying segments of the signal envelope that are above the threshold level to thereby identify corresponding segments of the digital audio recording of sounds from the subject as segments of the digital audio recording containing the particular sound events of interest.

### RELATED APPLICATIONS

The present application claims priority from Australian provisional patent application No. 2020902025, filed 18 Jun. 2020, the content of which is hereby incorporated herein by reference.

### TECHNICAL FIELD

The present invention relates to medical devices and more particularly to systems, devices and methods for detecting the presence of particular sound events, for example snore sounds or breath sounds such as wheezing, by analyzing a recording of a subject's sounds.

### BACKGROUND

Any references to methods, apparatus or documents of the prior art are not to be taken as constituting any evidence or admission that they formed, or form part of the common general knowledge.

Medical devices are known that include a transducer for converting sounds of a subject into electrical signals and which further include various assemblies that are responsive to the transducer and which in concert process the subject sounds to generate a prediction of the presence of respiratory maladies. Where a symptom of the malady is an event such as snoring or a breath sound such as wheezing then it would be advantageous if the medical device could be improved so that it is able to identify segments of the subject sounds that contain the event, as opposed to background noise for example. A medical device that is arranged to rapidly identify the event segments would make the device more efficient because the device could then be arranged to further process only segments containing the events and to quickly pass over other portions of the recording.

A number of approaches to identifying particular events of interest in subject sounds are known.

For example, one such technique is described in “Obstructive sleep apnea screening by integrating snore feature classes. Abeyratne U 2013 https://www.ncbi.nlm.nih.gov/pubmed/23343563” and another in “Dynamics of snoring sounds and its connection with obstructive sleep apnea. A. Alencar 2013”.

Both of these techniques require detecting the snore and breath sounds from a recording of the subject. However, the levels of the snore and breath sounds can be very low relative to the background noise level of the recording that is captured by a transducer. Additionally, pitch based techniques for detecting snores fail to detect breath sounds, which have no discernable pitch.

It may be the case that the signal to noise ratio of sounds associated with the event to the background noise is quite low. Consequently producing a medical device to achieve such an end is technically difficult. The recordings of the subject have very low volume levels, and many of the events of interest are buried within background noise.

There is a need for a solution to the problem of detecting one or more types of sound events of interest from a subject, where substantial background noise may be present, that is an improvement or at least a useful alternative to those solutions that are currently available.

### SUMMARY OF THE INVENTION

According to a first aspect of the invention there is provided a method for identifying segments of a digital audio recording of sounds from a subject, where the segments contain particular sound events of interest, the method comprising:

filtering the digital audio recording based on a characteristic frequency range of the sound events to produce a filtered digital audio signal;

processing the filtered digital audio signal to produce a corresponding signal envelope;

fitting a statistical distribution to the signal envelope;

determining a threshold level for the signal envelope based on the statistical distribution and a predetermined probability level; and

identifying segments of the signal envelope that are above the threshold level to thereby identify corresponding segments of the digital audio recording of sounds from the subject as segments of the digital audio recording containing the particular sound events of interest.

In an embodiment the digital audio recording is a recording of a digital audio signal that is comprised of a plurality of frames. For example, the digital audio signal may comprise a plurality of sequential, non-overlapping, frames. In one example the frames are of five-minute duration each though they may be shorter or longer.

In one embodiment the digital audio signal is made at a sampling rate of 44.1 kHz.

In an embodiment the method includes applying a first downsampling by which a sample rate of the digital audio recording is reduced by an integer factor to produce a first downsampled digital audio signal. For example, the digital audio signal may be downsampled by a factor of three from 44.1 kHz to 14.7 kHz so that the first downsampled audio signal has a sampling rate of 14.7 kHz.

In an embodiment the first downsampled digital audio signal is filtered in the characteristic frequency range to select for the sound events of interest to thereby produce a first downsampled and event-filtered digital audio signal.

In an embodiment the events of interest comprise breath sounds and wherein filtering the digital audio recording comprises applying a high pass filter.

In an embodiment the events of interest comprise snore sounds and wherein filtering the digital audio recording comprises applying a low pass filter.

In an embodiment processing the filtered digital audio signal to produce a corresponding signal envelope is implemented by an envelope detection procedure.

In an embodiment the envelope detection procedure includes applying an absolute value filter to the first downsampled and event-filtered signal to produce an absolute value filtered signal.

In an embodiment the absolute value filtered signal is filtered by a forward and reverse filter to produce a low pass filtered absolute value signal.

In an embodiment the method includes applying a second downsampling to the low pass filtered absolute value signal to produce the signal envelope, the signal envelope comprising a first signal envelope which is an estimate of amplitude of the audio recording.

In an embodiment applying the second downsampling comprises resampling from 14.7 kHz down to 100 Hz.

In an embodiment the method includes applying logarithmic compression to the first signal envelope to produce a second signal envelope that comprises a power estimate of the digital audio recording.

In an embodiment fitting the statistical distribution to the signal envelope comprises fitting the statistical distribution to the second signal envelope that comprises the power estimate.

In an embodiment fitting the statistical distribution to the signal envelope includes sorting samples making up the signal envelope into a number of bins to produce a histogram. For example, there may be 300 bins in one embodiment.

In an embodiment fitting the statistical distribution includes selecting a modal bin of the histogram, wherein the modal bin is a bin into which the greatest number of samples have been sorted.

In an embodiment bin number n contains samples in the range of n×step size+min to (n+1)×step size+min.

In an embodiment the statistical distribution comprises a Poisson distribution having a lambda parameter and fitting the statistical distribution includes setting the lambda parameter to the number of the modal bin.

In an embodiment determining the threshold level for the signal envelope based on the statistical distribution and the predetermined probability level comprises calculating a cumulative distribution function (CDF) in respect of the statistical distribution.

In an embodiment determining the threshold level for the signal envelope comprises finding a threshold bin, being a bin that corresponds to the predetermined probability, wherein the predetermined probability level comprises a probability level on the CDF.

In an embodiment determining the threshold level comprises setting the threshold level to a value from a range of magnitudes of samples in the threshold bin.

In an embodiment the threshold level is set to an upper limit of the range of magnitude of samples in the threshold bin.

In an embodiment a temporal filter is applied to cull segments that do not fall within a predetermined range of durations based on the events of interest.

In an embodiment the events of interest comprise snore sounds and wherein the range of durations is greater than 225 milliseconds and less than 4 seconds.

In an embodiment the method includes recording information indicating start and end times for each of the segments of the signal envelope that are above the threshold level in a non-volatile manner and in association with the digital audio recording.

According to a further aspect of the present invention there is provided an apparatus comprising a sound event identification machine configured to identify portions of a digital audio recording of a subject containing a particular sound events of interest, including:

a processor for processing the digital recording in accordance

a digital memory in data communication with the processor, the digital memory storing instructions to configure the processor, the instructions including instructions configuring the processor to:

filter the recording based on a characteristic frequency range of the sound events;

process the filtered recording to produce a corresponding signal envelope;

fit a statistical distribution to the signal envelope to thereby determine a threshold level corresponding to a predetermined probability level; and

identify segments of the signal envelope that are above the threshold to thereby identify corresponding segments of the digital audio recording as segments containing the particular sound events.

In an embodiment the apparatus includes a microphone that is configured to pick up sounds of the subject.

In an embodiment the apparatus includes an audio interface comprising a filter and an analog-to-digital converter configured to convert the sounds of the subject into a digital audio signal.

In an embodiment the apparatus is configured to store the digital audio signal as the digital audio recording in the digital memory accessible to the processor.

In an embodiment the apparatus includes a human-machine-interface.

In an embodiment the instructions stored in the digital memory include instructions that configure the processor to display information on the human-machine-interface including information identifying segments in the digital audio recording containing the events of interest.

In an embodiment the instructions stored in the digital memory include instructions that configure the processor to display information on the human-machine-interface including information indicating the event of interest.

In an embodiment the information that is displayed on the human-machine-interface includes information indicating a start time and an end time in respect of each of a number of segments identified to contain the event of interest.

In an embodiment the digital memory includes instructions that configure the processor to write the start and end times for each identified segment in a non-volatile manner to thereby tangibly label segments containing the events of interest in respect of the digital audio recording.

According to a further aspect of the present invention there is provided a machine-readable media bearing tangible, non-transitory instructions for execution by one or more processors to implement the method of claim 1.

According to another aspect of the present invention there is provided an apparatus for identifying portions of a digital recording of a subject containing particular sound events of interest, the apparatus comprising:

a transducer for converting sounds from the subject into a corresponding analogue electrical signal;

an analog-to-digital conversion assembly for generating a digital audio recording from the analogue electrical signal;

an events of interest filter for filtering the digital audio recording at a frequency characteristic of the particular sound events of interest to produce an events of interest filtered digital audio recording;

a signal envelope assembly for processing said filtered digital audio recording to produce a corresponding signal envelope;

a histogram generator assembly responsive to the signal envelope assembly for sorting digital samples comprising the signal envelope by their magnitudes into a plurality of bins and identifying a modal bin of the plurality of bins;

a statistical probability distribution generator responsive to the histogram generator and arranged to calculate a statistical probability distribution based on the identified modal bin and to determine a threshold level for the signal envelope from the statistical probability distribution and a predetermined probability level; and

an event identification assembly responsive to the statistical probability generator and arranged to identify segments of the signal envelope above the threshold level and tangibly identify corresponding segments of the digital recording as containing the events of interest.

According to a further aspect of the present invention there is provided a method for processing a digital audio recording of a subject, to identify one or more events of interest therein, the method comprising:

pre-processing the digital audio recording, including applying down-sampling and filtering thereto, to produce a corresponding signal envelope comprising a plurality of digital samples;

sorting the plurality of digital samples by their magnitudes into a plurality of bins;

determining a modal bin of the plurality of bins, the modal bin being a bin containing a greatest number of the plurality of digital samples having a magnitude within a range of the bin;

calculating a statistical probability distribution based on the identified modal bin;

determining a threshold bin being a bin corresponding to a predetermined probability level for the probability distribution;

setting a threshold level to a value from a range of the threshold bin; and

determining segments of the signal envelope above the threshold level to thereby tangibly identify corresponding segments of the digital audio recording containing the one or more events of interest.

In an embodiment the signal envelope comprises a power estimate signal for the digital audio recording.

In an embodiment the signal envelope comprises an amplitude estimate signal for the digital audio recording.

In an embodiment the down-sampling and filtering of the digital audio recording includes applying a low pass filter to the digital audio recording in forward and reverse directions.

In an embodiment the down-sampling and filtering of the digital audio recording includes applying a high pass filter wherein the events of interest comprise breath sounds of the subject.

In an embodiment the down-sampling and filtering the digital audio recording includes applying a low pass filter wherein the events of interest comprise snore sounds of the subject.

In an embodiment the calculating of the statistical probability distribution based on the identified modal bin comprises calculating a Poisson distribution using an index of the modal bin as a lambda parameter of the Poisson distribution.

According to further aspect of the present invention there is provided an apparatus for identifying portions of a digital recording of a subject containing a particular sound event, the apparatus including:

a processor for processing the digital recording in accordance with instructions stored in a digital memory accessible to the processor, the instructions including instructions for the processor to implement a method for detecting segments of the digital recording containing particular events of interest.

It should be appreciated that features or characteristics of any aspect or embodiment thereof may be incorporated into any other aspect unless logic dictates otherwise.

### BRIEF DESCRIPTION OF THE DRAWINGS

Preferred features, embodiments and variations of the invention may be discerned from the following Detailed Description which provides sufficient information for those skilled in the art to perform the invention. The Detailed Description is not to be regarded as limiting the scope of the preceding Summary of the Invention in any way. The Detailed Description will make reference to a number of drawings as follows:

FIG. 1 is a flowchart of a method according to a preferred embodiment.

FIG. 2 is a graph of a frame of a digital audio signal recorded during performance of the method.

FIG. 3 depicts an initial twenty second portion of the signal shown in FIG. 2.

FIG. 4 is a graph of a downsampled, compressed and filtered waveform produced during performance of the method comprising an amplitude estimate corresponding to the signal of FIG. 3.

FIG. 4A is a graph of the log of the signal illustrated in FIG. 4, comprising a power estimate corresponding to the signal of FIG. 3.

FIG. 5 is a histogram of the waveform of FIG. 4 that is generated during performance of the method with a Poisson distribution curve based on the histogram and CDF for the Poisson distribution also shown.

FIG. 6 is a graph corresponding to FIG. 4A with an event threshold level shown thereon.

FIG. 7 is a graph of the waveform of FIG. 3 showing segments identified to contain events of interest corresponding to the segment times indicated in FIG. 6.

FIG. 8 is a block diagram of an apparatus for identifying events of interest in an audio signal of a subject according to an embodiment.

FIG. 9 is a block diagram of an event identification machine according to an embodiment.

FIG. 10 is an external view of the machine of FIG. 9 in a stage of use.

FIG. 11 is an external view of the machine of FIG. 9 in a subsequent stage of use.

### DETAILED DESCRIPTION OF PREFERRED EMBODIMENTS

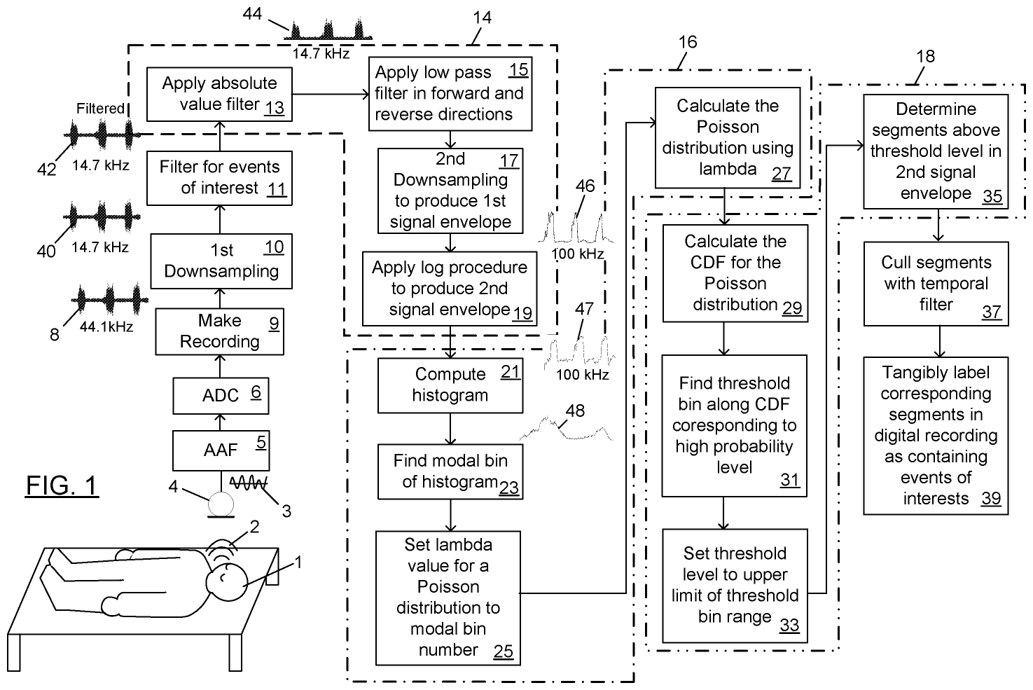

A method for automatic event detection according to a preferred embodiment of the present invention will be described with reference to the flowchart of FIG. 1.

In overview, the method involves processing a digital audio recording to identify segments of the recording containing particular sound events of interest. The digital audio recording is processed according to a number of processes including filtering the digital audio recording, at box 11, and processing the filtered digital audio recording to produce a corresponding signal envelope, as indicated by dashed box 14. A statistical distribution, which is typically the Poisson distribution but which could be another statistical distribution such as the gamma distribution, is then fitted to the signal envelope, as indicated by dashed box 16. A threshold level is then determined, as indicated by dashed line 18, in respect of the signal envelope. The threshold level is determined based on the statistical distribution and a predetermined probability level. Segments of the signal envelope that are above the threshold level are then identified for example as start and finish times of each such segment, to thereby also identify corresponding segments of the digital audio recording that contain the particular sound events of interest. For example the sound events of interest may be sounds such as snoring, or wheezing or breathing sounds.

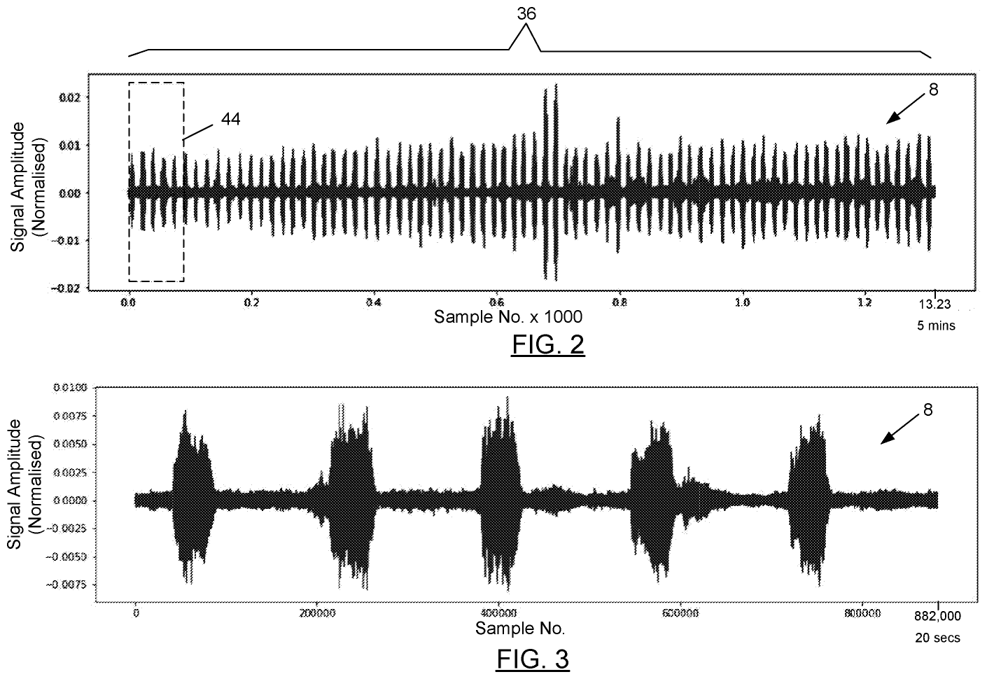

Initially, prior to commencement of the method that has been discussed in overview, a transducer in the form of microphone 4 converts analog, air-borne sound wave 2 from subject 1 into a corresponding analogue electrical signal 3. Analog electrical signal 3 is subsequently processed at box 5 by an anti-aliasing filter then at box 6 by an analog-to-digital converter to form a corresponding digital audio signal At box 9, the digital audio signal from ADC 6 is stored in an electronic data storage assembly, such as a digital memory, as a digital audio recording. The digital audio recording is subsequently retrieved in the form of a digital audio signal 8 and processed by the following boxes of the flowchart of FIG. 1 in accordance with a preferred embodiment of the method. In the present embodiment digital audio signal 8 is comprised of a plurality of sequential, non-overlapping, five minute frames. FIG. 2 shows a single frame 36 of the digital audio signal 8 which at a sampling rate of 44.1 kHz is comprised of 44,100×5×60=13,230 samples. FIG. 3 shows in more detail the first twenty seconds (identified as item 44 in FIG. 2) of the digital audio signal 8 in frame 36.

At box 10 the digital audio signal 8 is retrieved from the digital memory and is subjected to a first downsampling from its original sample rate of 44.1 kHz to 14.7 kHz so that the number of samples per second are reduced by a factor of three. Downsampling in this fashion produces a first downsampled digital audio signal 40 that contains fewer samples for subsequent processing. Since the digital audio signal will be processed to detect a noise floor threshold, rapidly moving transients are non-essential and so the downsampling does not result in a loss of accuracy. At box 11 the first-downsampled digital audio signal 40 is filtered by applying a bandpass, or low pass or high pass filter to frequency select for the sound event of interest that is to be tangibly identified in the digital audio signal 8. For example, at box 11 a 1000 Hz high pass filter may be applied to the first downsampled digital audio signal 40 if it is the case that the particular sound event of interest is a breath sound. Alternatively, if the particular sound event of interest comprises snore sounds then a low pass filter at 1000 Hz may be applied.

Boxes 13 to 19 implement an envelope detection procedure 14 that pre-processes signal 42 prior to application of subsequent steps for identifying events of interest, as will be explained.

At box 13 an absolute value filter is applied to the downsampled, event-filtered audio signal 42, which flips all negative samples to positive to produce a corresponding absolute value filtered audio signal 44. For example, where the ADC at box 7 samples at 16 bit resolution then each sample will have an integer amplitude value in the range of −32,768 to +32,767 amplitude steps. The absolute value filtering at box 13 inverts the sign of the negative amplitude samples so that all samples then take an integer amplitude value in the range of 0 to +32,767.

The absolute value-filtered audio signal 44 is then passed to a 7 Hz low pass forward and reverse filter at box 15. In order for the reverse filter part of the procedure at box 15 to operate it is necessary for the absolute value-filtered audio signal 44 to be stored in a digital memory. The forward-reverse filter effects low pass filtering without impacting on the phase of the content of interest.

At box 17 a 2^{nd} downsampling operation is applied to the filtered absolute value signal 44. The 2^{nd} downsampling operation resamples from 14.7 kHz down to 100 Hz.

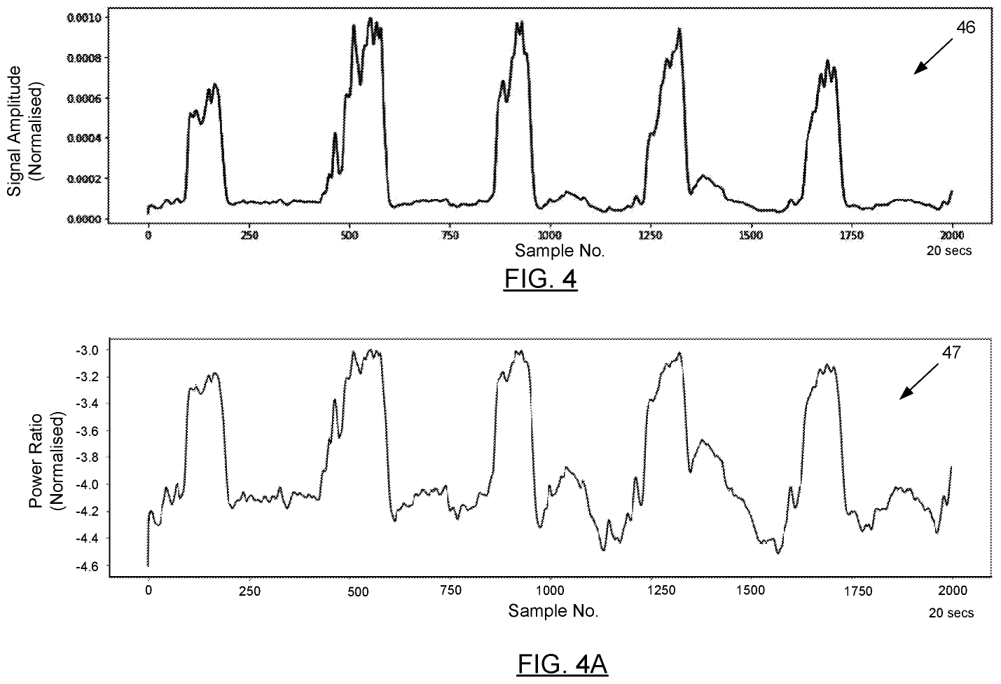

FIG. 4 depicts the forward-reverse filtered signal generated at box 15 which comprises a first signal envelope signal or more simply a “signal envelope” that corresponds to the original recorded signal 8 and which is an estimate of the amplitude of the recorded signal 8.

Logarithmic compression is then applied at box 19 to amplitude estimate signal 46 to reduce large input signal variations. Prior to applying the logarithmic compression, samples having power ratio values less than 10⁻⁵ are adjusted up to a magnitude of 10⁻⁵ in order to limit the range of values subsequent to applying the logarithmic compression. The absolute value filter at box 13, low pass filter at box 15, downsampling at box 17, and logarithmic compression at box 19 produce a second signal envelope 47 corresponding to the original digital recording 8. The second signal envelope 47 is a power estimate signal, being an estimate of the power of the original digital audio signal 8.

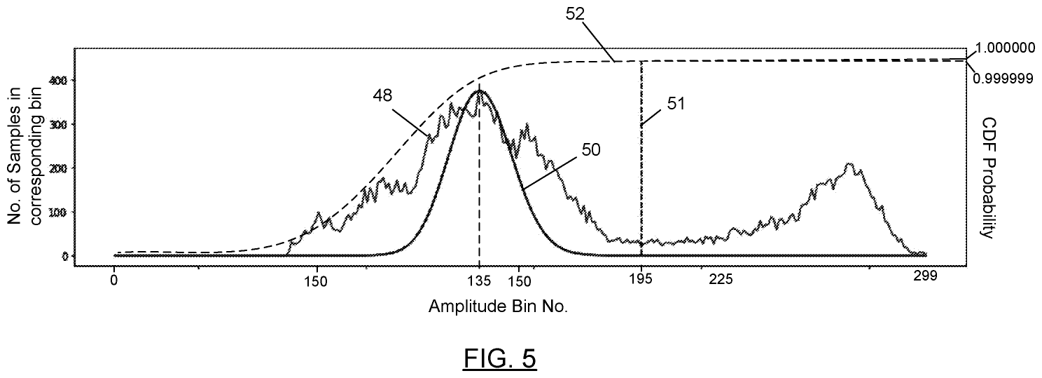

At box 21 a histogram 48 (FIG. 5) of sample magnitudes over a frame, e.g. five minutes, of the second signal envelope 47 is calculated. The histogram 48 is generated by sorting the 2000 samples that comprise the second signal envelope (power estimate signal) 47 by their magnitudes, each into one of 300 magnitude bins.

It will of course be realized that in other embodiments a different downsampling ratio might be used so that the five minute frame will comprise fewer or greater than 2000 samples. Furthermore, whilst a five minute frame is preferred a frame might be longer or shorter and its length may be adjusted based on the downsampling ratio and the number of bins to be used. It will also be realized that more or less than 300 bins might be used though 300 is a preferred number of bins that has been found to work well for the presently described downsampling ratio and frame length.

Whilst it is preferred that the compute histogram procedure in box 21 is performed on the second signal envelope 47, which is the power estimate signal, it could instead be performed on the 1^{st} signal envelope, i.e. the amplitude estimate signal 46. The reason for performing the log procedure at box 19 is to avoid rapid amplitude changes of the amplitude signal envelope which would make the subsequent histogram and statistical distribution fitting steps, which will be explained, less reliable.

For the second signal envelope, which comprises the power estimate signal 47 depicted in FIG. 4A, each sample falls within a range of −5.0 to −2.9 (being the minimum and maximum power estimates in the frame). The samples making up the power estimate signal 47 which may be sorted into 300 bins (sequentially indexed as “0” to “299”) with a step size of (Max−Min)/Num bins, i.e. (−2.9−−5.0)/300=approximately 0.007. Bin number n contains samples in the range of n×step size+min to (n+1)×step size+min. For example, bin number 150 contains samples in the power estimate range of 150×0.007+−5.0 to (150+1)×0.007+−5.0, i.e. −3.95 to −3.94.

At box 23 the modal bin of the histogram 48 is selected. The modal bin is the bin into which the greatest number of samples in the frame have been sorted. In the example illustrated in FIG. 5 almost 400 samples of the 30,000 samples comprising the second signal envelope 47 over the five minute frame, have been sorted into magnitude bin 135, so that it contains more samples than any other bin and thus bin number 135 is the modal bin. Magnitude bin 135 contains samples of the second signal envelope with magnitudes representing power estimates for the original recorded signal 8 in the range of (135×0.007+−5.0) to ((135+1)*0.007+−5.0) , i.e. −4.053 to −4.046.

At box 25 the lambda parameter of a Poisson distribution is set to the number of the modal bin for fitting the distribution to the histogram. That is, lambda=135 since bin 135 contains the most samples. At box 27 a Poisson distribution with lambda=135 is calculated. The Poisson distribution is graphically illustrated as line 50 in FIG. 5 fitted over histogram 48. At box 29 a cumulative distribution function (CDF) is calculated for the Poisson distribution. The CDF is shown on the graph of FIG. 5 as curve 52.

At box 31 a threshold bin of the histogram 48 is found, being a bin that corresponds to a very high probability level on the CDF 52. The probability level is set to be very high because it is desired to be able to identify audio segments that contain events of interest to a high level of confidence. In the case illustrated in FIG. 5 the probability level has been set to 0.999999 of a total probability under the CDF curve of 1. The 0.999999 probability level is found to correspond to bin number 195 as indicated by the dashed vertical line 51. Bin number 195, which is thus the threshold bin, contains samples with magnitudes representing power estimates in the range of 195×0.007+−5.0 to (195+1)*0.007+−5.0, i.e. −3.632 to −3.625. At box 33 the upper limit of the magnitude of the samples in bin 195, i.e. −3.625 is set to a threshold level for the second signal envelope, i.e. the power estimate signal 47.

At box 35 segments of the second signal envelope 47 containing samples with magnitudes that are all above the threshold level are determined by comparing each sample making up the second signal envelope 47 to the threshold level.

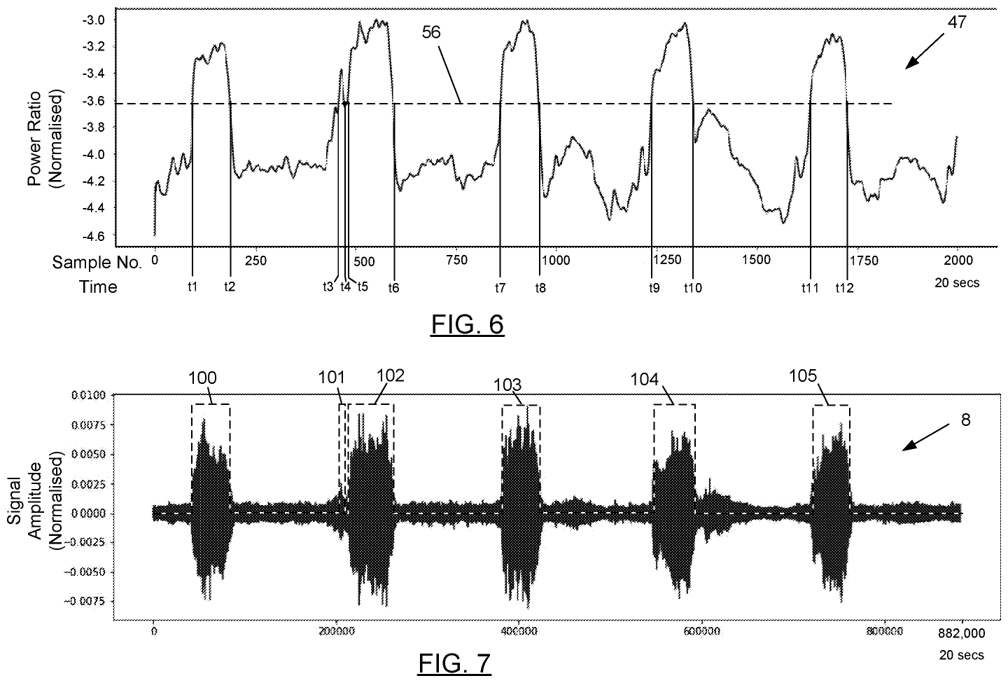

FIG. 6 shows the threshold level 56 superimposed on the second signal envelope (i.e. the power estimate signal) 47.

It can be seen that the second signal envelope exceeds threshold in the following above-threshold segments, [t1,t2]; [t3,t4]; [t5,t6]; [t7,t8]; [t9,t10]; and [t11,t12].

A simple temporal filter is applied at box 37 to select above threshold segments that potentially correspond to sleep sounds, being events of interest, on the basis that the events must be of longer duration than 225 ms and less duration than 4 s. Accordingly, interval 101 is discarded leaving the intervals identified as containing the particular sleep event as intervals 100, 102, 103, 104 and 105.

Those segments are deemed to have a very high likelihood of corresponding to segments in the original waveform 8 that contain samples for the particular event that was filtered for as event of interest in box 11.

At box 39 the above-threshold level segments are then tangibly labelled in respect of the initial digital audio signal 8 as intervals 100, 101, 102, 103, 104 and 105. By “tangibly” labelled it is meant that the start and end times, or equivalent information, for each of the above-threshold segments is recorded in a non-volatile manner in association with the recording of the digital audio sound so that the above-threshold segments can be readily identified and processed further as necessary.

The digital audio signal 8, along with labels identifying segments in the signal that contain the events of interest can then be further processed. For example, if the events of interest are snore sounds then the segments labelled as containing snores can then be processed using prior art methods to determine if the snore sounds are indicative of sleep apnea. Similarly, if the events of interest are wheezing sounds then the digital audio signal and the labels identifying segments containing the wheezing sounds can then be processed to determine if the wheezing is indicative of asthma for example.

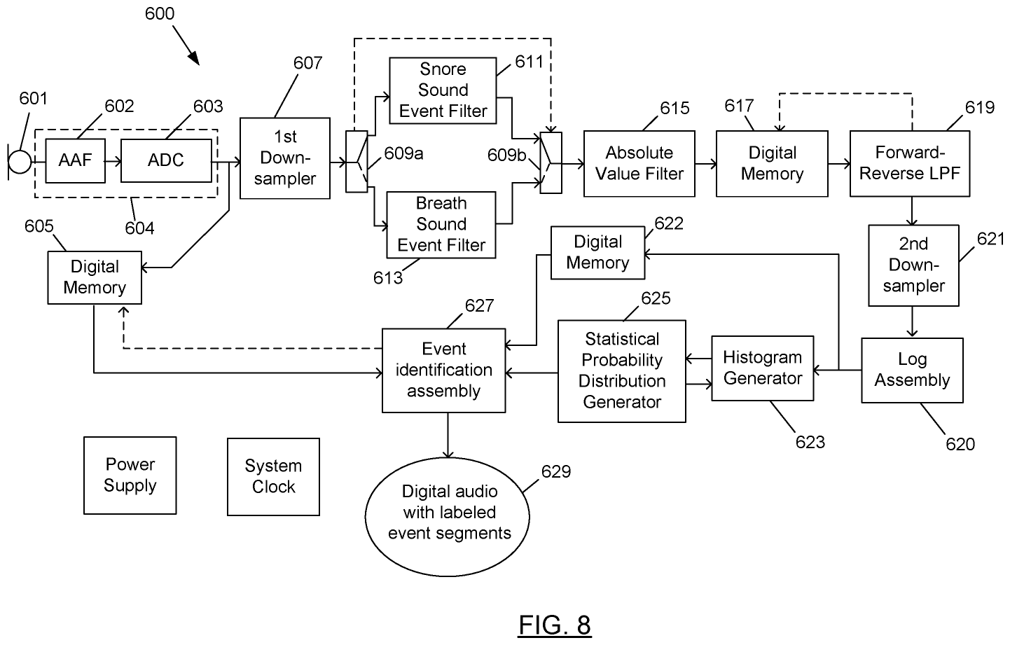

FIG. 8 is a block diagram of an apparatus 600 for identifying portions of a digital recording of a subject containing a particular sound event in accordance with the method that has previously been described. Apparatus 600 comprises a transducer in the form of microphone 601 for converting sounds 3 from the subject 1 into a corresponding analogue electrical signal 8 (FIG. 2).

The microphone 601 is connected to an analog-to-digital conversion assembly 604 for generating a digital audio recording from the analogue electrical signal. The analog-to-digital conversion assembly 604 is comprised of anti-aliasing-filter 602 and analog-to-digital converter 603 which produces a corresponding digital signal to the sounds 3 from the subject 1. In the present embodiment the analog-to-digital conversion assembly 1 is arranged to produce a 44.1 kHz sample rate signal at 16 bit resolution. It will be realized that other sampling rates and bit resolutions may also be used in other embodiments of the present invention.

An output port of the analog-to-digital conversion assembly 604 provides the digital signal in five minute frames to a digital memory 605. The output port is also connected to a 1^{st} Downsampler 607 which is arranged to downsample from 44.1 kHz to 14.7 kHz.

Signal from the 1^{st} Downsampler 607 proceeds through either Snore Sound Event Filter 611 or Breath Sound Event Filter 613 to absolute value filter 615 depending on the setting of ganged switches 609a and 609b. Snore Sound Event Filter 611 and Breath Sound Event Filter 613 are respectively a 1000 Hz cutoff Low Pass filter for selecting snore sound events and a 1000 Hz High Pass filter for selecting breath sound events. Both filters are 2nd Order Butterworth High/Low Pass IIR filters with a cutoff of 1000 hz.

Output of switch 609b is coupled to an absolute value filter 615 which is arranged to reverse the sign of all negative samples in the filtered digital audio signal. The absolute value filter 615 is in turn coupled to a digital memory 617 which stores frames of filtered digital signal from the absolute value filter 615.

A Forward-Reverse Low Pass Filter 619 is coupled to the Digital Memory 617 for filtering the stored signal in both forward and reverse directions with a 2nd Order Butterworth Low Pass IIR, cutoff of 7 Hz filter. The signal is filtered twice, forwards and then backwards to preserve the phase.

A second downsampler 619 is coupled to an output side of the Forward-Reverse LPF 619 to perform downsampling of the signal to 100 Hz, which results in an amplitude estimate signal such as signal 46 of FIG. 4.

A log amplifier assembly 620 is coupled to an output side of the 2^{nd} downsampler 621. The log amplifier assembly generates a power estimate signal, for example signal 47 of FIG. 4A. A digital memory 622 is coupled to the log amplifier assembly 620 and stores frames of the power estimate signal.

A histogram generator assembly 623 is coupled to an output side of the second downsampling assembly. The histogram generator assembly 623 is arranged to sort digital samples comprising the power estimate signal by their magnitudes into a plurality of magnitude bins and generate a signal indicating a modal magnitude bin of the plurality of magnitude bins and a Poisson distribution lambda value for a pre-set high probability threshold for the distribution.

A statistical probability distribution generator 625 is provided that is responsive to the histogram generator 623 and is arranged to calculate a statistical probability distribution based on the identified modal magnitude bin. Histogram generator assembly 623 and distribution generator 625 may be implement by one or more FPGAs or microcontrollers for example configured to calculate a distribution such as a Poisson distribution using the signal indicating a modal magnitude bin as the lambda parameter for the Poisson distribution.

An event identification assembly 627 is provided that is responsive to the statistical probability generator 625 and which is arranged to tangibly identify segments of the digital recording in digital memory 605, containing the particular event being segments containing samples above a background noise sample value for a predetermined probability level.

The event identification assembly 627 can either insert meta-data codes into a file 629 storing the original audio signal or alternatively it may write a file containing a sequence of time intervals which effectively label the segments of the audio signal containing the events of interest.

As an alternative to a Poisson distribution, the Inventors have also tested other log-normal distributions from which they believe that a normal distribution will also work. However, the Poisson distribution is preferred since it has a technical advantage of being simple to fit as there is only one parameter (i.e. the Lambda parameter) to estimate which is straightforward to extract from the histogram.

The requirement for the chosen distribution is that it can be fit to the histogram of the samples and the noise samples follow that distribution.

For example if the distribution family was chosen to be gamma, then a fitting function such as one provided by scipy at the following link https://docs.scipy.orq/doc/scipy/reference/generated/scipy.stats.gamma.html may be used to estimate the parameters of the gamma distribution.

In use the apparatus 600, which can be implemented in a sufficiently small housing for holding by hand, is held a few centimeters from the subjects face by an operator. Where a lengthy recording is required the apparatus 600 may be mounted to a tripod for the duration of the recording. The operator configures ganged switches 609a, 609b to select either the snore sound event filter 611 or the breath sound event filter 613 depending on the type of event of interest to be identified in the recorded sound signal. As the recording progresses a digitised sound recording is stored in digital memory 605 which will typically comprise a recording medium such as an SD card. At the same time the signal is also variously downsampled and filtered by the various blocks 607 to 621 of apparatus 600 as previously described to produce a power estimate signal at the output side of the 2^{nd} Downsampler 621. Histogram generator 623 then sorts the samples making up the power estimate signal by their magnitudes into a plurality of bins to determine a modal bin. Probability distribution generator 625 uses the number of the modal bin as a lambda parameter for a Poisson distribution in order to calculate a corresponding Poisson distribution and from a CDF of that distribution identify a bin corresponding to a very high probability of containing samples above noise floor. Event identification assembly 627 is is responsive to the statistical probability generator 625 is arranged to tangibly identify segments of the digital recording, which is stored in digital memory 605, containing the particular event being segments containing samples above the background noise sample value for a predetermined probability level.

It will be understood that in an embodiment a method is provided for identifying segments of a recording of a signal comprising sounds from a subject containing particular sound events of interest. The method involves filtering the recording based on a characteristic frequency range of the sound events. For example a lower frequency range is used where the sound events of interest are predominantly lower frequency sounds such as snores, in contrast to a higher frequency range for other sounds such as breath sounds. The method then includes processing the filtered recording to produce a corresponding power estimate signal, which is an estimate of the power of the original recorded audio signal. The method then involves fitting a statistical distribution to the power estimate signal, for example a Poisson distribution, and determining a noise floor threshold level from the distribution using a high probability level that the noise threshold level is indeed above a noise floor of the signal in respect of the events of interest. Segments of the recording of sounds from the subject are then identified as segments that are above the noise floor threshold level.

The method quickly identifies segments in recording of the patient sound that are very likely to contain the events of interest so that time and processing power can be spent on further analysing those segments without wasting time on processing segments that do not contain the sound events of interest.

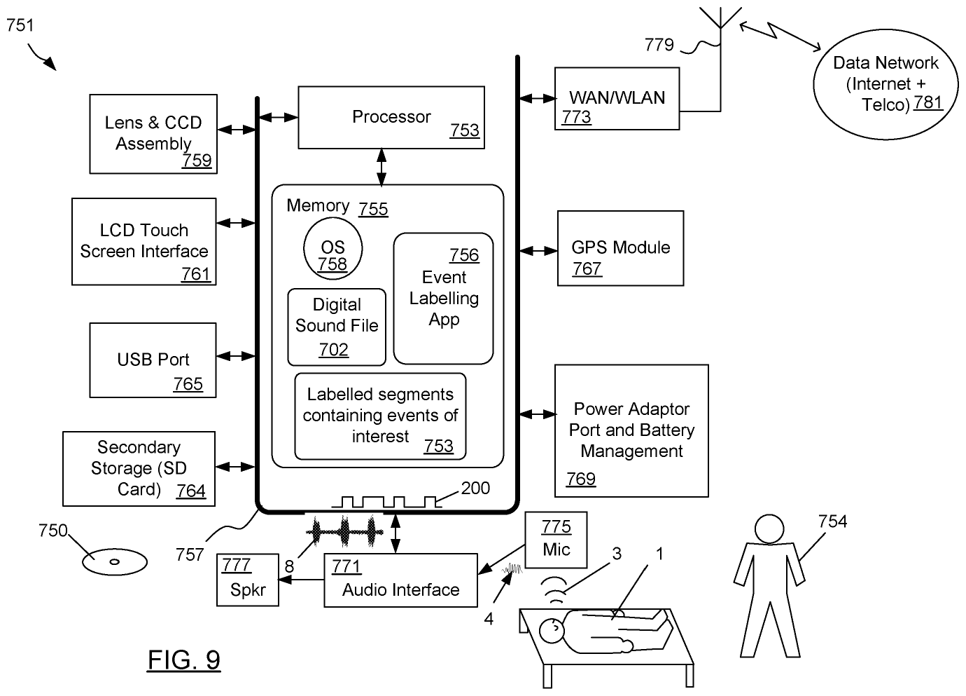

FIG. 9 is a block diagram of a sound event identification machine, 751 according to another embodiment of the invention for identifying and labelling segments of a sound recording that contain events of interest, such as snore or wheeze sounds for example. In the presently described embodiment the apparatus is implemented using one or more processors, microphone and memory of a smartphone. The sound event identification machine 751 includes at least one processor 753 that accesses an electronic memory 755. The electronic memory 755 includes an operating system 758 such as the Android operating system or the Apple iOS operating system, for example, for execution by the processor 753. The electronic memory 755 also includes a sound event identification software product or “App” 756 according to a preferred embodiment of the present invention. The cough identification App 756 includes instructions that are executable by the processor 753 in order for the sound event identification machine 751 to process sounds 702 from a subject 1 in accordance with the method of FIG. 1. During its operation the processor 753 under command of App 756 processes the sounds 702 and presents a list of segments containing sound events of interest to an operator 754 by means of LCD touch screen interface 761. The identified sound events can then be further processed if desired. The App 756 may be provided as tangible, non-transitory, machine readable instructions borne upon a computer readable media such as optical or magnetic disk 750 for reading by disk drive coupled to USB port 765. Alternatively the App may also be downloaded from a remote file server via WAN/WLAN interface 773.

The processor 753 is in data communication with a plurality of peripheral assemblies 759 to 773, as indicated in FIG. 9, via a data bus 757 which is comprised of metal conductors along which digital signals 200 are conveyed between the processor and the various peripherals. Consequently, if required the sound event identification machine 751 is able to establish voice and data communication with a voice and/or data communications network 781 via WAN/WLAN assembly 773 and radio frequency antenna 779.

The machine 751 also includes other peripherals such as Lens & CCD assembly 759 which effects a digital camera so that an image of subject 752 can be captured if desired along with the location at which the image was taken using data from GPS module 767. Machine 751 also includes a power adaptor port and battery management assembly 769 for powering the machine. A LCD touch screen interface 761 is provided that acts as a human-machine interface and allows the operator 754 to read results and input commands and data into the machine 751. A USB port 765 is provided for effecting a serial data connection to an external storage device such as a USB stick or for making a cable connection to a data network or external screen and keyboard etc. A secondary storage card 764 is also provided for additional secondary storage if required in addition to internal data storage space facilitated by memory 755.

Audio interface 771 couples a microphone 775 to data bus 757 and includes anti-aliasing filtering circuitry and an Analog-to-Digital sampler to convert the analog electrical waveform 4 from microphone 775 (which corresponds to subject sound wave 3) to a digital audio signal 8 which is stored as a recording in digital sound file 702 in memory 755 and for processing by processor 753 under control of App 756. For example, the processor may be a Snapdragon 865 processor manufactured by Qualcomm Corporation, though other and lesser powered processors will also be suitable. The audio interface 771 is also coupled to a speaker 777. The audio interface 771 includes a Digital-to-Analog converter for converting digital audio into an analog signal and an audio amplifier that is connected to speaker 771 so that audio 702 recorded in memory 755 or secondary storage 764 can be played back for listening by operator 754.

The machine 751 is programmed with App 756 so that it is configured to identify segments containing events of interest, such as wheezing or snoring, in the recording of the subject sound.

As previously discussed, although the sound event identification machine 751 that is illustrated in FIG. 9 is provided in the form of smartphone hardware that is uniquely configured by App 756 it might equally make use of some other type of computational device such as a desktop computer, laptop, or tablet computational device or even be implemented in a cloud computing environment wherein the hardware comprises a virtual machine that is specially programmed with App 756.

An embodiment of the procedure that cough identification machine 751 uses to identify segments containing events of interest in a recording 702 of subject 752, and which comprises instructions that make up App 756 is illustrated in the flowchart of FIG. 1 which has previously been described.

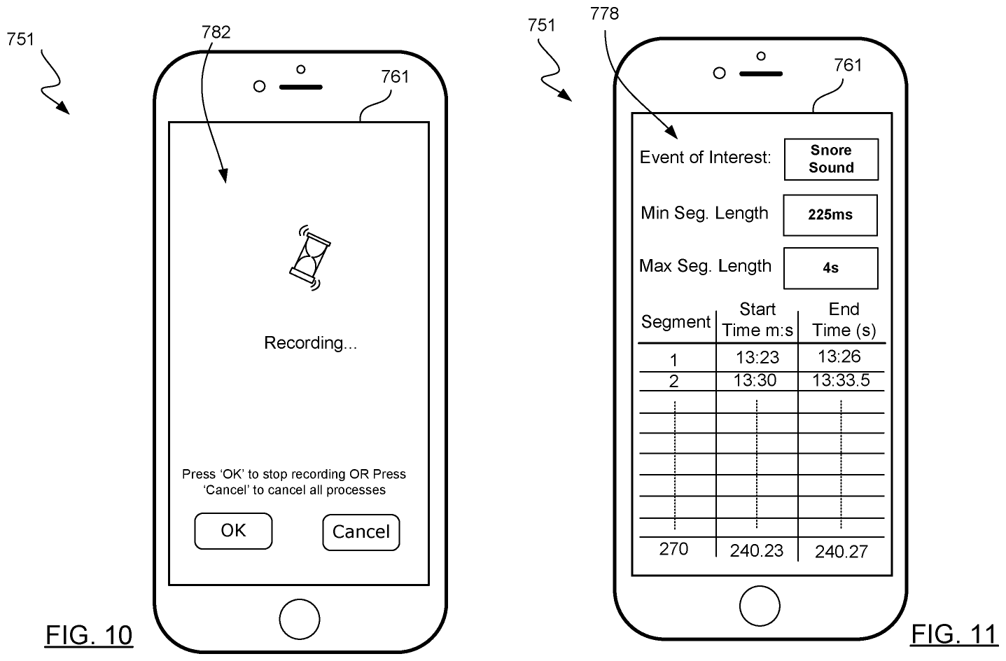

In use operator 754 or subject 3, selects App 756 from an app selection screen generated by OS 758 on LCD touchscreen interface 761. In response to that selection the processor 753 displays a screen such as screen 782 of FIG. 10 to prompt the operator 754 to operate machine 751 to commence recording sound 3 from subject 752 via microphone 775 and audio interface 771. The audio interface 771 converts the sound into digital signals 200 which are conveyed along bus 757 and recorded as one or more digital files 702 by processor 753 in memory 755 and/or secondary storage SD card 764. In the presently described preferred embodiment the recording should proceed for a duration that is sufficient to include a number of sound events of interest.

After the recording has finished the processor 753 under control of instructions comprising the App 756, which implement the method of FIG. 1, processes the recording 702 and identifies segments in the recording containing events of interest. The identified segments may then be displayed on screen 778 which, in the present example identifies 270 segments that contain sound events of interest along with the start and end times of each segment. Processor 753 under control of App 756 also writes the identified segment numbers and start and end times in a non-volatile manner to a file 753 that may also contain the sound wave recording in order to tangibly label the events of interest in respect of the sound wave recording.

The method that is implemented by processor 753 in combination with the instructions comprising event labelling app 756 quickly identifies segments in the recording 702 of the subjects sound that are very likely to contain the events of interest. Consequently time and processing power can be spent on further analysing those segments if desired without wasting time on processing segments that do not contain the sound events of interest.

In compliance with the statute, the invention has been described in language more or less specific to structural or methodical features. The term “comprises” and its variations, such as “comprising” and “comprised of” is used throughout in an inclusive sense and not to the exclusion of any additional features.

It is to be understood that the invention is not limited to specific features shown or described since the means herein described comprises preferred forms of putting the invention into effect. The invention is, therefore, claimed in any of its forms or modifications within the proper scope of the appended claims appropriately interpreted by those skilled in the art.

Throughout the specification and claims (if present), unless the context requires otherwise, the term “substantially” or “about” will be understood to not be limited to the value for the range qualified by the terms.

Any embodiment of the invention is meant to be illustrative only and is not meant to be limiting to the invention. Therefore, it should be appreciated that various other changes and modifications can be made to any embodiment described without departing from the scope of the invention.

1. A method for identifying segments of a digital audio recording of sounds from a subject, where the segments contain particular sound events of interest, the method comprising:
filtering the digital audio recording based on a characteristic frequency range of the sound events to produce a filtered digital audio signal;
processing the filtered digital audio signal to produce a corresponding signal envelope;
fitting a statistical distribution to the signal envelope;
determining a threshold level for the signal envelope based on the statistical distribution and a predetermined probability level; and
identifying segments of the signal envelope that are above the threshold level to thereby identify corresponding segments of the digital audio recording of sounds from the subject as segments of the digital audio recording containing the particular sound events of interest.

2. The method of claim 1, including applying a first downsampling by which a sample rate of the digital audio recording is reduced by an integer factor to produce a first downsampled digital audio signal.

3. The method of claim 2, wherein the first downsampled digital audio signal is filtered in the characteristic frequency range to select for the sound events of interest to thereby produce a first downsampled and event-filtered digital audio signal.

4. The method of claim 3, wherein the events of interest comprise breath sounds and wherein filtering the digital audio recording comprises applying a high pass filter.

5. The method of claim 3, wherein the events of interest comprise snore sounds and wherein filtering the digital audio recording comprises applying a low pass filter.

6. The method of claim 3, wherein processing the filtered digital audio signal to produce a corresponding signal envelope is implemented by an envelope detection procedure that includes applying an absolute value filter to the first downsampled and event-filtered signal to produce an absolute value filtered signal.

7. (canceled)

8. The method of claim 6, wherein the absolute value filtered signal is filtered by a forward and reverse filter to produce a low pass filtered absolute value signal.

9. The method of claim 8, including applying a second downsampling to the low pass filtered absolute value signal to produce the signal envelope, the signal envelope comprising a first signal envelope which is an estimate of amplitude of the audio recording.

10. (canceled)

11. The method of claim 9, including applying logarithmic compression to the first signal envelope to produce a second signal envelope that comprises a power estimate of the digital audio recording.

12. The method of claim 11, wherein fitting the statistical distribution to the signal envelope comprises fitting the statistical distribution to the second signal envelope that comprises the power estimate.

13. The method of claim 1, wherein fitting the statistical distribution to the signal envelope includes sorting samples making up the signal envelope into a number of bins to produce a histogram and wherein fitting the statistical distribution includes selecting a modal bin of the histogram, wherein the modal bin is a bin into which the greatest number of samples have been sorted.

14. (canceled)

15. The method of 13, wherein the statistical distribution comprises a Poisson distribution having a lambda parameter and fitting the statistical distribution includes setting the lambda parameter to the number of the modal bin.

16. The method of claim 1, wherein determining the threshold level for the signal envelope based on the statistical distribution and the predetermined probability level comprises calculating a cumulative distribution function (CDF) in respect of the statistical distribution and wherein determining the threshold level for the signal envelope comprises finding a threshold bin, being a bin that corresponds to the predetermined probability level, wherein the predetermined probability level comprises a probability level on the CDF.

17. (canceled)

18. The method of claim 16, wherein determining the threshold level comprises setting the threshold level to a value from a range of magnitudes of samples in the threshold bin.

19. (canceled)

20. The method of claim 18, wherein a temporal filter is applied to cull segments that do not fall within a predetermined range of durations based on the events of interest.

21. (canceled)

22. (canceled)

23. An apparatus comprising a sound event identification machine configured to identify portions of a digital audio recording of a subject containing a particular sound events of interest, including:
a processor for processing the digital recording;
a digital memory in data communication with the processor, the digital memory storing instructions to configure the processor, the instructions including instructions configuring the processor to:
filter the recording based on a characteristic frequency range of the sound events;
process the filtered recording to produce a corresponding signal envelope;
fit a statistical distribution to the signal envelope to thereby determine a threshold level corresponding to a predetermined probability level; and
identify segments of the signal envelope that are above the threshold to thereby identify corresponding segments of the digital audio recording as segments containing the particular sound events.

24. The apparatus of claim 23, including a microphone that is configured to pick up sounds of the subject.

25. The apparatus of claim 23, including an audio interface comprising a filter and an analog-to-digital converter configured to convert the sounds of the subject into a digital audio signal, wherein the apparatus is configured to store the digital audio signal as the digital audio recording in the digital memory accessible to the processor.

26. (canceled)

27. The apparatus of claim 23, including a human-machine-interface, wherein the instructions stored in the digital memory include instructions that configure the processor to display information on the human-machine-interface including information identifying segments in the digital audio recording containing the events of interest.

28. (canceled)

29. (canceled)

30. (canceled)

31. The apparatus of claim 27, wherein the digital memory includes instructions that configure the processor to write a start and an end time for each identified segment in a non-volatile manner to thereby tangibly label segments containing the events of interest in respect of the digital audio recording.

32. A machine-readable media bearing tangible, non-transitory instructions for execution by one or more processors to implement the method of claim 1.
